(19) United States
(12) Patent Application Publication
(10) Pub. No.: US 2020/0088736 A1
(43) Pub. Date: Mar. 19, 2020

(54) FLUID COMPOSITION, METHOD FOR PREPARING THE COMPOSITION AND USE

(71) Applicant: Lifecare AS, Bones (NO)

(72) Inventor: Rune FRISVOLD, Bones (NO)

(21) Appl. No.: 16/494,191

(22) PCT Filed: Mar. 1, 2018
(86) PCT No.: PCT/NO2018/050055

§ 371 Date: Sep. 13, 2019

(30) Foreign Application Priority Data
Mar. 15, 2017 (NO) 20170385

(51) Int. Cl. G01N 33/66

(52) U.S. Cl. CPC G01N 33/66

(57) ABSTRACT

A composition including a carbohydrate-binding molecule, the carbohydrate molecule being a lectin; a lectin-binding molecule, the lectin-binding molecule being a polysaccharide; at least one chloride salt of a divalent metal ion, in which the lectin to polysaccharide molar ratio is from 3:1 to 1:1. A method of preparing the above composition, and the usage of the composition in carbohydrate sensing systems.

### TECHNICAL FIELD

The present application relates to fluid compositions, in special active fluid compositions which may be used in glucose sensor systems, for example a glucose sensor which relies on osmotic pressure as sensing principle.

### BACKGROUND

Commonly glucose measurement is based on manual point sample sensors instrumentation (finger-pricking). However, devices capable of conducting continuous blood glucose measurements would provide the most complete picture of the glucose variations during the course of the day and prevent the onset of dangerous events by for example trigger an alarm function when the blood glucose moves beyond what are considered safe levels. This is especially important when persons are sleeping or not being able to look after themselves. Although the continuous blood glucose measurement instrumentation is considered as the most effective method of monitoring glucose, the transcutaneous nature of the sensor patches, combined with limited sensor lifetimes and long start-up periods, has meant that the single use sensor for manual point sampling remains the most common.

There are some drawbacks to the manual point sampling method. The persons often experience pain and discomfort with the manual point sample devices, which might in turn compromise such self-testing regimes. Incomplete numbers of measurements taken during the course of a day may result in that the average person with diabetes spending periods during the days in a hyperglycaemic or hypoglycaemia state. Both these conditions are potentially dangerous and can contribute to vascular damage, mental confusion and even death.

The benefits of existing continuous sensing technologies come with major drawbacks and disadvantages, and hence there are currently no real commercial alternatives to the manual point sample method. Existing continuous glucose measurements technologies systems are inconvenient, complicated and costly. There are no alarm functions or digital memory for storing the data. The existing systems also have a limited operational lifetime and require frequent calibrations using external point sample meters.

Detecting glucose by the principle of osmotic pressure holds promise of a glucose sensing technology that is suitable for both miniaturisation and long term continuous monitoring in vivo without causing patient discomfort or reducing quality of life. An osmotic sensor for measuring blood glucose is described in the PhD Thesis “Osmotic sensor for blood glucose monitoring applications”, by Olga Krushinitskaya, Department of Micro-and Nanosystems Technology, Vestfold University College, August 2012. The project in this PhD work addressed the technological aspect of developing a novel glucose sensor that was capable of tracking glucose continuously through the recording of osmotic pressure, based on the principle of utilizing the diffusion of water down its own concentration gradient, which enables an inherently simple sensor design in which the generated pressure is a function of the glucose concentration.

The osmotic sensor developed in the said project was based on the osmotic pressure generated by the competitive bonding between the sugar binding lectin Concanavalin A (ConA) and the long chained polysaccharide dextran, which forms a large macromolecular complex. Lectins are a group of proteins that have special binding sites for carbohydrates, and the ConA attaches strongly to glucose. The studies in the above Thesis exploited the osmotic effect generated by the competitive bonding of ConA and dextran in the presence of glucose. As the concentration of glucose is increased, more of the larger ConA-dextran macromolecular complexes are split up into the smaller ConA-glucose and free dextran “sub units”. In this manner the number of free particles inside the sensor is increased as a function of glucose, leading to a corresponding rise in the osmotic pressure, see FIG. 1A-B.

This process is reversible and as the glucose concentrations falls, the Con A reattaches back to the dextran forming a large macromolecular complex from the Con A and dextran “sub units”. The corresponding decrease in the number of free particles triggers the osmotic pressure to fall.

Granted European patent EP 1 631 187 B1 discloses a sensor for in vivo measurement of osmotic changes. The sensor is an invasive sensor which can be implanted subcutaneously, and specially an invasive sensor comprising at least one differential pressure-transducer that measures the pressure difference between two fluid volumes confined by, in one end the at least one differential pressure-transducer, and in the other end osmotic membranes.

The sensor described in EP 1 631 187 B1 can be utilized to monitor any changes within the in chemistry in vivo. The type of solutes and their concentration observed in vivo gives a tremendous amount of information regarding the physiology of the body, and its condition. By measuring the composition for instance in the interstitial fluid (ISF), a lot of information can be obtained regarding de-hydration of the body and different diseases: diabetes, kidney functions, etc. Also normal variations for instance in lactate concentrations caused by physical activity can be monitored.

In addition to the substances mentioned above, which can change the osmolality in the body, one can also find substances which by medication give an osmotic contribution in the body fluid.

Measurement of glucose in ISF is becoming recognized as an alternative to measuring the glucose directly in the blood. The glucose measurement in blood is associated with several drawbacks. It needs a sample of blood, drawn from the body. Even though the equipment has become more sensitive, and therefore requires less blood, the process is associated with pain and the number of tests typically limited to less than 10 per day. It is also known that large variations in measured values can be caused by the measurement procedure.

Embodiments and details of the sensor are shown in FIGS. 1-6B in EP 1 631 187 B1 and in the description paragraphs [0022] to [0054].

The current invention concerns a composition which can be used as an active fluid in continuous glucose sensing technology without the above described disadvantages. It was found that an optimal active fluid composition should have low viscosity and the viscosity should be substantially independent from the glucose concentration. The present compositions of the active fluids provide faster response time compared to previous known compositions due to lower fluid viscosity and optimal composition. The handling of the active fluids are also easier due to the lower viscosity. Further, the optimal compositions result in measurable osmotic pressure changes by the pressure sensor employed, i.e. the system sensitivity is highly improved. The active fluids chemistry exhibit reproducible concentrations, show longtime stable responses at room temperature (>3 months), and the concentrations are stable at 37° C.

Thus, the active fluid composition according to the invention has advantageous effect on
sensor lifetime; due to high fluid stability, no precipitation of the active compounds, and no leakage of active compounds through membrane;
measurement response time; due to low fluid viscosity;
measurement response asymmetry; due to viscosity is substantially independent from the glucose concentration,
measurement sensitivity; due to optimal concentration and choice of compounds.

### SUMMARY OF THE INVENTION

In a first aspect the present invention provides an active fluid composition for use as an active fluid in a continuous glucose sensor comprising a carbohydrate-binding molecule, the carbohydrate-binding molecule being a lectin; a lectin-binding molecule, the lectin-binding molecule being a polysaccharide; at least one chloride salt of a divalent metal ion, and optionally glucose, wherein the said lectin to said polysaccharide molar ratio may be from 3:1 to 1:1.

In a first embodiment the lectin to polysaccharide molar ratio in the said composition is from 2:1 to 1:1. In a second embodiment the lectin to polysaccharide molar ratio in the said composition is 3:1, 2:1 or 1:1.

In a third embodiment the polysaccharide in the said composition may be dextran or other polysaccharide, having a molecular weight 10-100 kDa, e.g. 10-70 kDa. The dextran may have a molecular weight of 10 kDa, 40 kDa or 70 kDa.

In a fourth embodiment the concentration of said lectin in the said composition may be 0.2-5 mM, 0.5-3 mM or more specific 1-1.5 mM.

In a fifth embodiment the lectin in the said composition is Concanavalin A (ConA).

In a sixth embodiment the concentration of the polysaccharide in the said composition is 0.2-5 mM, 0.5-3 mM or more specific 1-1.5 mM.

In a seventh embodiment the concentration of ConA in the composition is 0.2-5 mM, more specific 0.5-3 mM, 1-1.5 mM, 1.5 mM or 1 mM based on the monomer concentration, and the concentration of dextran is 0.2-5 mM, more specific 0.5-3 mM, 1-1.5 mM, 1.5 mM or 1 mM. In an eight embodiment the ConA concentration in the composition is 1 mM based on the monomer concentration, and the dextran concentration is 1 mM. In a ninth embodiment the said ConA concentration is 1.5 mM based on the monomer concentration, and the dextran concentration is 1.5 mM.

In a tenth embodiment the composition, according to any of the above embodiments, may further comprise an aqueous buffer solution with pH 7.0-7.8. The buffer may be a Tris buffer or HEPES.

In a specific embodiment according to the tenth embodiment, the aqueous buffer solution contains 10-120 mM, for instance 100 mM, of a Tris buffer. In another specific embodiment according to the tenth embodiment the aqueous buffer solution in said composition contains 1-40 mM, or 1-20 mM, of HEPES buffer. The aqueous buffer solution, according to the tenth embodiment, may have a pH 7.2-7.6, for instance 7.35-7.5 or 7.4-7.5.

Verification of pH in the buffer solution may be performed at room temperature or 37° C. using a reference pH meter. Other temperatures may also be utilized depending on the intended use of the composition.

In an eleventh embodiment of the said composition, according to any of the above embodiments, the at least one chloride salt of a divalent metal ion is chosen from MgCl₂, CaCl₂ and MnCl₂. In a specific embodiment of the eleventh embodiment, the at least one chloride salt of a divalent metal ion, or a combination thereof is dissolved in the said aqueous buffer solution giving concentrations 1-10 mM MgCl₂, 1-10 mM CaCl₂ and 1-10 mM MnCl₂. The aqueous solution may also comprise NaCl giving an isotonic solution.

In a specific embodiment according to the eleventh embodiment, the said aqueous buffer solution comprises at least one of 10 mM MgCl₂, 10 mM CaCl₂ and 10 mM MnCl₂ or a combination thereof and 150 mM NaCl, and optionally 20-40 mM glucose. In another specific embodiment according to the eleventh embodiment, the aqueous buffer solution contains 10 mM MgCl₂, 10 mM CaCl₂, 150 mM NaCl, and 30 mM glucose. The presence of glucose minimizes ConA-dextran binding, keeping the viscosity of the solution low and making handling easier.

In the composition to the present invention, according to any of the above embodiments, the water used in the aqueous buffer solution is reverse osmosis water, deionized water or distilled water.

In a specific embodiment a composition according to present invention has the following composition
1.0 mM or 1.5 mM ConA based on the monomer concentration,
1.0 mM or 1.5 mM dextran 40 or dextran 70,
Tris buffer 100 mM, pH 7.4-7.5,
10 mM MgCl₂, 10 mM CaCl₂ and 150 mM NaCl,
30 mM glucose
wherein the said lectin to said polysaccharide molar ratio is 1:1.

In a further embodiment of the composition, according to any of the above embodiments, the continuous glucose sensor measures the changes in the concentrations of glucose in fluids in vitro or in vivo by detecting osmotic pressure differences.

In another embodiment of the composition, according to any of the above embodiments, the continuous glucose sensor is tracking glucose concentrations continuously through the recording of osmotic pressure, for measuring glucose concentrations in vitro or in vivo.

In a second aspect the present invention provides a method for preparing a composition suitable for use as an active fluid in a continuous glucose sensor, according to any of the above embodiments, comprising the following steps
(i) preparing an aqueous buffer solution having pH from 7.0 to 7.8, comprising at least one chloride salt of a divalent metal, and optionally glucose,
(ii) either (a) providing the desired amount of a lectin-binding molecule, the lectin-binding molecule being a polysaccharide, to which the aqueous buffer solution prepared in step (i) is added and left stirring until homogenized and dissolved, and thereafter providing the desired amount of a carbohydrate-binding molecule, the carbohydrate-binding molecule being a lectin to which the said buffer solution containing said lectin-binding molecule is added and left stirring until homogenized and dissolved, or
   (b) providing the desired amount of a carbohydrate-binding molecule, the carbohydrate-binding molecule being a lectin, to which the aqueous buffer solution prepared in step (i) is added and left stirring until homogenized and dissolved, and thereafter providing the desired amount of a lectin-binding molecule, the lectin-binding molecule being a polysaccharide, to which the said buffer solution containing said lectin is added and left stirring until homogenized and dissolved.

In a first embodiment of the method, the solutions in step (ii) is stirred for 2-48 hours, for instance 6-36 hours, or 12-24 hours for dissolution and homogenization of the said lectin and said polysaccharide in the aqueous buffer solution.

In a second embodiment of the method, the molar ratio of lectin to polysaccharide ratio is from 3:1 to 1:1, e.g. from 2:1 to 1:1, 3:1, 2:1 or 1:1.

In a third embodiment of the method, the concentration of lectin is 0.2-5 mM, or more specific 0.5-3 mM, for example 1-1.5 mM. In a fourth embodiment of the method, the concentration of lectin is 1 mM. In a fifth embodiment of the method, the concentration of lectin is 1.5 mM. According to any of the embodiments of the method, the said lectin may be ConA.

In a sixth embodiment of the method, the polysaccharide is 0.2-5 mM, or more specific 0.5-3 mM, for example 1-1.5 mM. In a seventh embodiment of the method, the concentration of polysaccharide is 1 mM. In an eight embodiment of the method, the concentration of said polysaccharide is 1.5 mM. According to any of the embodiments of the method, the said polysaccharide may be dextran or other lectin-binding polysaccharide, having a molecular weight 10-100 kDa, e.g. 10-70 kDa. The said dextran may have a molecular weight 10 kDa, 40 kDa or 70 kDa.

In a ninth embodiment of the method, the method for preparing the buffer solution in the above step (i) may include, preparing an aqueous buffer solution having pH 7.0 to 7.8, at least one of 1-10 mM MgCl₂, 1-10 mM CaCl₂ and 1-10 mM MnCl₂ or a combination thereof, and optionally glucose.

In a tenth embodiment of the method, the buffer is chosen from a Tris buffer or HEPES buffer. In a specific embodiment of the tenth embodiment of the method, the said aqueous buffer solution may contain 10-120 mM of a Tris buffer, for instance 100 mM Tris buffer. In another specific embodiment of the tenth embodiment of the method, the said aqueous buffer solution may contain 1-40 mM HEPES buffer, for instance 1-20 mM HEPES buffer. The buffer solution, according to the tenth embodiment of the method, may have a pH 7.35-7.5, for instance 7.4-7.5. The pH may be verified by using a pH reference meter, the verification is performed at room temperature or at 37° C. Other temperatures may also be utilized depending of the use of the composition.

In an eleventh embodiment of the method, the said method the aqueous buffer solution may contain at least one of 10 mM MgCl₂, 10 mM CaCl₂ and 10 mM MnCl₂ or a combination thereof and 150 mM NaCl, and optionally 20-40 mM glucose. In a twelfth embodiment of the method, the said aqueous buffer solution contains 10 mM MgCl₂, 10 mM CaCl₂ and 150 mM NaCl, and 30 mM glucose.

In any of the above embodiments of the method, the active fluid may be degassed in order to minimize formation of bubbles.

In a specific embodiment of the method, the fluid composition is prepared by the following steps
preparing an aqueous buffer solution containing 100 mM Trizma buffer, 10 mM MgCl₂, 10 mM CaCl₂, 150 mM NaCl, and 30 mM glucose,
verifying pH 7.4-7.5,
the desired quantity of dextran is weighed and buffer solution is added and left overnight while stirring, giving a dissolved and homogenized dextran solution,
the desired quantity of ConA is weighed out and dextran solution is added and left under stirring for 24 hours before use.

The aqueous buffer solution may be degassed in order to minimize formation of bubbles. After the degassing the volume should be checked and made up if necessary.

In a third aspect of the invention, the composition according to present invention may be used as an active fluid in a sensor for measuring the changes in the concentrations of carbohydrates in fluids in vitro or in vivo by detecting osmotic pressure differences. In an embodiment the active fluid composition according to present invention may be used for measuring blood glucose concentrations in vitro or in vivo. The said composition may be used as an active fluid in an osmotic glucose sensor that is capable of tracking glucose concentrations continuously through the recording of osmotic pressure, for measuring glucose concentrations in vitro or in vivo.

In the context of present invention the terms “composition”, “active fluid”, “fluid composition”, “reference fluid” are all expressions referring to the composition according to the present invention.

### BRIEF DESCRIPTION OF THE DRAWINGS

FIG. 1: Illustration of sensing principle; FIG. 1A. Low glucose, low pressure difference; FIG. 1B. High glucose, high pressure difference. FIG. 1C. Macrocell in-vitro; FIG. 1D. Linear correlation between osmotic pressures and glucose levels.

FIG. 2: Viscosity as a function of glucose concentration for dextran 10 kDa, 40 kDa and 70 kDa. ConA concentration is equal to 3 mM, dextran concentration is 0.5 mM. Please note the y-logarithmic scale.

FIG. 3: Viscosity as a function of glucose concentration for dextran 10 kDa, 40 kDa and 70 kDa. ConA concentration is equal to 1.5 mM, dextran concentration is 0.5 mM. Please note the y-logarithmic scale.

FIG. 4: Viscosity as a function of glucose concentration for dextran 10 kDa, 40 kDa and 70 kDa. ConA concentration is equal to 1 mM, dextran concentration is 1 mM. Please note the y-logarithmic scale.

FIG. 5: Viscosity as a function of glucose concentration for two dextrans, 40 kDa and 70 k. ConA and dextran concentrations are equal to 1.5 mM.

FIG. 6: Viscosity as a function of glucose concentration for dextran 40 kDa, with two different ConA and dextran concentrations (1 and 1.5 mM).

FIG. 7: Viscosity as a function of glucose concentration for dextran 70 kDa, with two different ConA and dextran concentrations (1 and 1.5 mM).

FIG. 8: An example curve showing changes between 2 mM glucose solution and 30 mM glucose solution. Note the strong fast spike followed by a slow drift in the opposite direction to reach a stable equilibrium.

### DETAILED DESCRIPTION OF THE INVENTION

The object of present invention is to provide a composition which can be used as an active fluid in continuous glucose sensing technology. This object have been achieved by the composition comprising

a carbohydrate-binding molecule, the carbohydrate-binding molecule being a lectin;
a lectin-binding molecule, the lectin-binding molecule being a polysaccharide;
at least one chloride salt of a divalent metal ion,
wherein the said lectin to said polysaccharide molar ratio is from 3:1 to 1:1.

In the above composition the carbohydrate-binding molecule is a lectin. Lectins are carbohydrate-binding proteins, macromolecules that are highly specific for sugar moieties. Concanavalin A (ConA) is a lectin originally extracted from the jack-bean, Canavalia ensiformis. It is a member of the legume lectin family. It binds specifically to certain structures found in various sugars, glycoproteins, and glycolipids, mainly internal and nonreducing terminal α-D-mannosyl and α-D-glucosyl groups. It is known to exhibit long term chemical stability at physiological body temperatures. The configuration of ConA depends on the pH. Monomeric sub-units are formed at pH 4-6 in the presence of 2-propanol, dimeric at pH 4.5-6.5, whereas the tetrameric structure is formed at a pH higher than 7. The size of the Con A monomer is approximately 42×40×39 Å. The molecular weight of one such sub-unit range from 25500 Da to 27000 Da, depending on the literature reference that is consulted. One sub-unit contains one binding site for certain structures found in sugars, e.g. glucose or mannose, and considering the tetrameric structure, such a molecule would have a total of 4 binding sites. The affinity towards carbohydrates is governed by a metal ion binding site that both activates Con A for saccharide binding as well as modulating its stability. Ca²⁺, Mg²⁺ and Mn²⁺ may be used for activating the ConA, the Mn²⁺ ion can be replaced by Co²⁺, Ni²⁺, Zn²⁺ and Cd²⁺.

Further, in the above composition the lectin-binding molecule is a polysaccharide. In present work dextran has been used as lection-binding molecule. Dextran is a complex branched glucan (polysaccharide made of many glucose molecules) composed of chains of varying lengths (from 3 to 2000 kDa. The straight chain consists of α-1,6 glycosidic linkages between glucose molecules, while branches begin from α-1,3 linkages. The branching of dextran can be from 0.5-60%, with the solubility decreasing as the branching is increased. Dextran is synthesized from sucrose by certain lactic acid bacteria, the best-known being Leuconostoc mesenteroides and Streptococcus mutans. The chemical formula for dextran is (from Mehvar et al. Dextran for trageted and sustained delivery of therapeutic and imaging agents. Journal of Controlled release, 2000. 69: p. 1-25.)

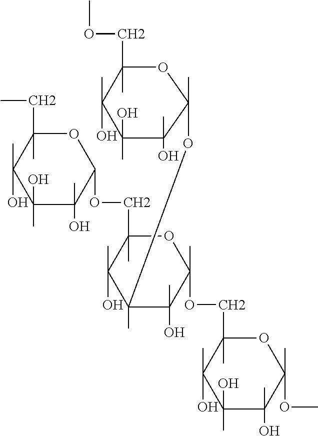

The above composition further includes at least one chloride salt of a divalent metal ion. The at least one chloride salt of a divalent metal ion is chosen from MgCl₂, CaCl₂ and MnCl₂. The at least one chloride salt, or a combination thereof is dissolved in an aqueous buffer solution giving concentrations 1-10 mM MgCl₂, 1-10 mM CaCl₂ and 1-10 mM MnCl₂. The aqueous solution may also comprise NaCl giving an isotonic solution.

In the composition according to present invention the buffer used for preparing the aqueous buffer solution may be chosen from Tris buffer, other names tris(hydroxymethyl)aminomethane or THAM, is an organic compound with the formula (HOCH2)3CNH2. Tris buffer is also known as Trizma®, which is a trademark belonging to Sigma-Aldrich®. Tris is used as a component of buffer solutions, such as in TAE (Tris-acetate-EDTA) and TBE (Tris-barate-EDTA) buffer. TAE buffer is a buffer solution containing a mixture of Tris base, Acetic acid and EDTA. TBE buffer is a buffer solution containing a mixture of Tris base, Boric acid and EDTA. The Tris-buffered saline (TBS) is a buffer used in some biochemical techniques and is isotonic and non-toxic. TBS contains Tris and NaCl. The pKₐ of Tris buffers is dependent on temperature, the pKₐ declines approximately 0.03 units per degree Celsius rise in temperature.

or

HEPES buffer (4-(2-hydroxyethyl)-1-piperazineethanesulfonic acid; IUPAC name: 2-[4-(2-hydroxyethyl)piperazin-1-yl]ethanesulfonic acid) which is a zwitterionic organic chemical buffering agent. The buffer solution may be a combination of HEPES acid/Na HEPES salt and NaCl

In the composition according to the present invention glucose may further be added for the purpose of reducing the viscosity if the active fluid composition. At low glucose concentrations, dextran molecules are cross-linked by ConA bonds, forming an extremely viscous solution. When the glucose concentration increases, dextran molecules are partially replaced by glucose at the binding sites of ConA. As a result, the network ConA-dextran is weakened and the viscosity of the active fluid decreases.

NaCl may be added to the active fluid composition to produce an isotonic solution.

The water used for preparing the active fluid, that is the buffer solution is a purified type of water such as reverse osmosis water, deionized or distilled water.

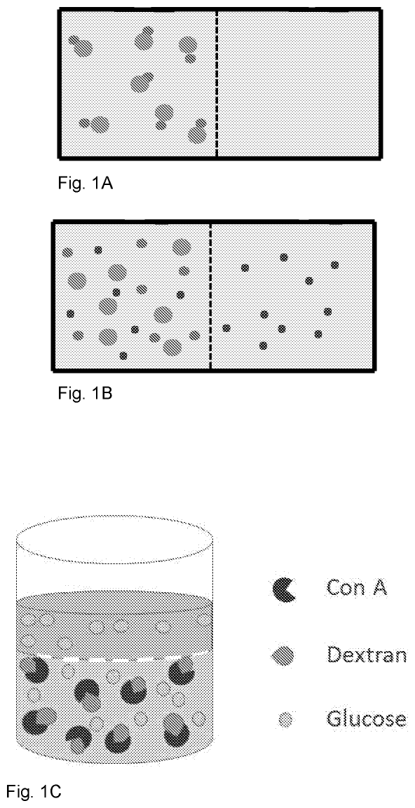

The sensing principle of an implantable glucose sensor relies on osmotic pressure variations between a reagent chamber and the solution, see FIGS. 1A-D. Detection of glucose is based on the competition between glucose and a polysaccharide (dextran) to bind to a receptor, the lectin Concanavalin A (ConA). ConA and dextran are present in the reagent chamber in an “active fluid” and are not to exit through the nanoporous membrane. At low glucose concentration, the pressure difference in the reagent chamber is low. Glucose molecules and small ions will pass through the pores of the membranes. Glucose molecules which enter the reagent chamber compete with dextran to bind to ConA proteins. As the concentration of glucose increases the release of dextran from ConA results in an increase of osmotic pressure that can be used to quantify the concentration of glucose. This process is reversible and as the glucose concentrations falls, the ConA reattaches back to the dextran forming a large macromolecular complex from the ConA and dextran “sub units”. The corresponding decrease in the number of free particles triggers the osmotic pressure to fall.

The osmotic pressure H of an ideal solution of low concentration can be approximated using the Morse equation:

π=iMRT

where i is the dimensionless Van't Hoff factor, M is the molarity, R the gas constant and T the temperature of the chamber.

FIG. 1D shows the linear correlation between osmotic pressures and glucose levels, accurate also at hypo and hyper glycemic levels.

The inventors found that lowering the viscosity of the composition would consequently reduce the asymmetry and response time of the sensor. In order to diminish the viscosity of the active fluid, the following parameters were explored while varying the glucose concentration in the solutions:
Molecular weight of the dextran (10 kDa, 40 kDa or 70 kDa)
Ratio of ConA to dextran
Initial concentration of ConA.

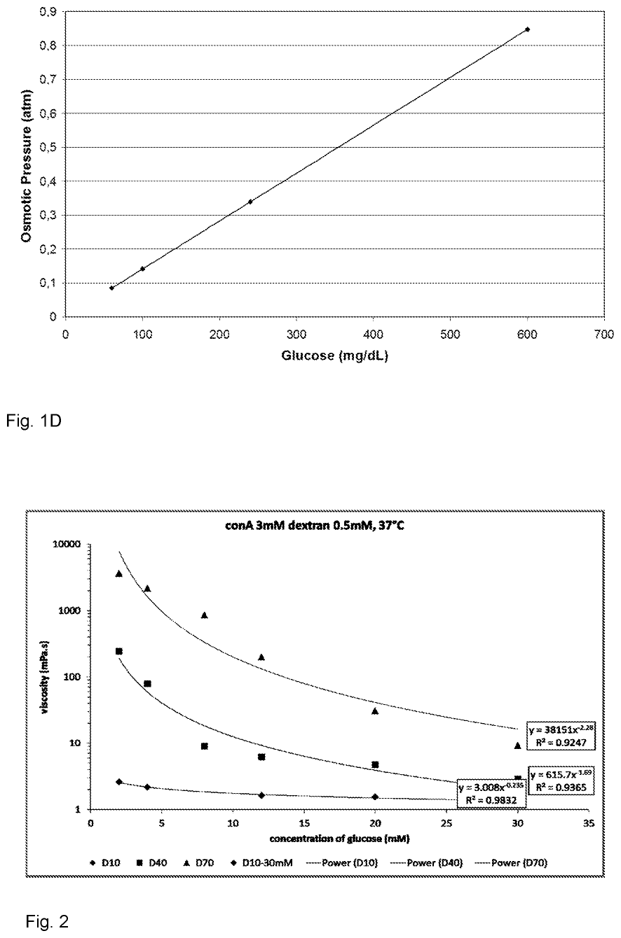

FIG. 2 shows viscosity as a function of glucose concentration for dextran 10 kDa, 40 kDa and 70 kDa, the molar ratio between ConA to dextran was 6:1, using initial concentrations respectively 3 mM and 0.5 mM. This active fluid composition was considered as a “baseline” for the viscosity measurements.

Lowering the ConA concentration and more specifically, the ConA to dextran concentrations ratio, was expected to decrease the number of intermolecular bonds between dextran and ConA, thus lowering the viscosity of the system. To investigate this effect, the ConA to dextran was reduced from 6:1 to 3:1, using the following concentrations ConA 1.5 mM, dextran 0.5 mM and glucose range 2 to 30 mM.

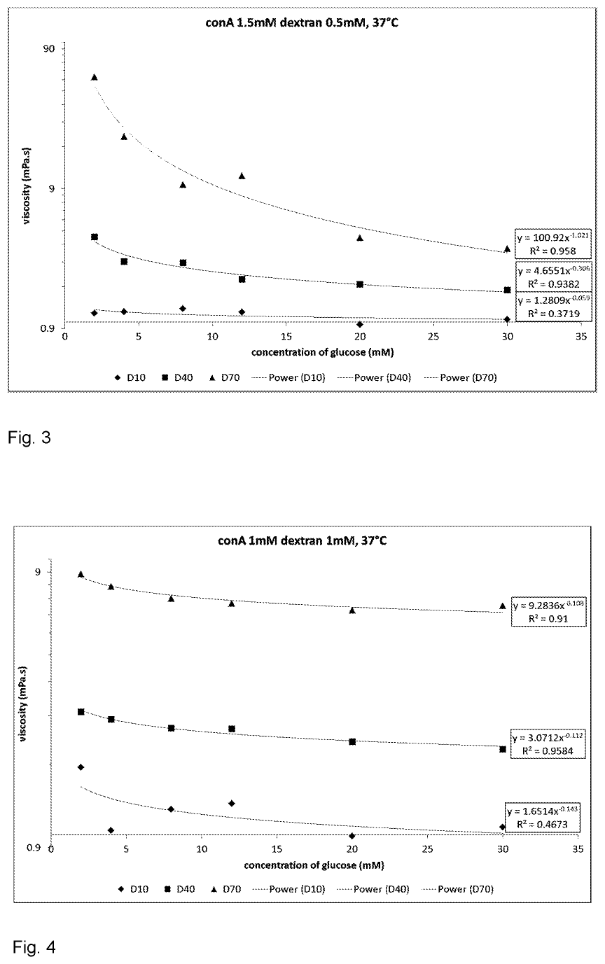

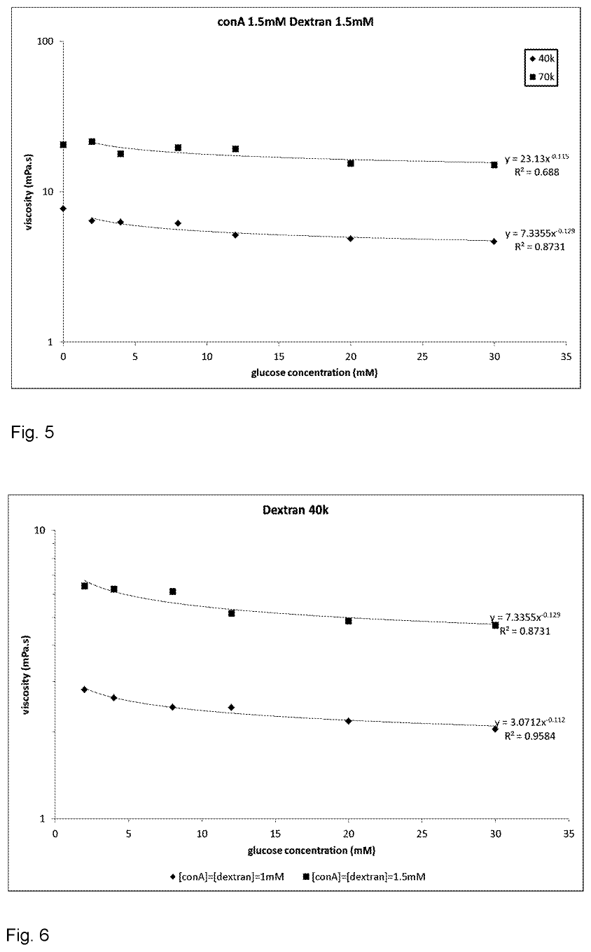

FIG. 3 clearly shows that the viscosity of each system is decreased by one to two orders of magnitude when the ConA concentration is divided by two (all other parameters are kept constant). This is expected to have an extremely significant effect on the response time and the asymmetry of the response with increasing or decreasing glucose concentration.

Decreasing the molar ratio of ConA to dextran to 1:1 was shown to improve the amplitude of the response of the sensor. Lowering the number of intermolecular bonds between ConA and dextran also lowered the viscosity. The inventors chose to test a slightly higher concentration of dextran (1 mM instead of 0.5 mM) since it was also shown to improve the amplitude of the response and the sensitivity of the sensor. An active fluid with a ConA and dextran concentrations of 1 mM showed an amplitude of response approximately three times larger than the amplitude of response of the “baseline” active fluid described above.

By keeping the molar concentrations of dextran and ConA equal (at 1 mM), the viscosity was further decreased by an order of magnitude when compared to 3:1 molar ratio of ConA to dextran, see FIG. 4. Furthermore, the influence of the glucose concentration on viscosity is lessened. This decreased the asymmetry of the response times to ascending and descending glucose concentrations.

For characterisations, each solution was stirred during 24 hours before viscosity measurements. A Brookfield DV-II+Pro viscometer was used for all measurements. The viscometer drives a spindle through a calibrated spring which is immersed in the active fluid. The viscous drag of the fluid against the spindle is measured by the spring deflection, which is measured with a rotary transducer. The measurement range is determined by the rotational speed of the spindle, the size and shape of the spindle, the container where the spindle is rotating in, and the full scale torque of the calibrated spring. The viscosity appears in units of centipoise (shown “cP”). One centipoise is equal to 1 mPa·s in USI.

To control the temperature (T) at which the measurements were done, the viscometer was equipped with a water bath which can be set at the chosen T. All the viscosity measurements were performed at 37° C., with a 5 min. waiting period to ensure stabilisation of the temperature in the active fluid.

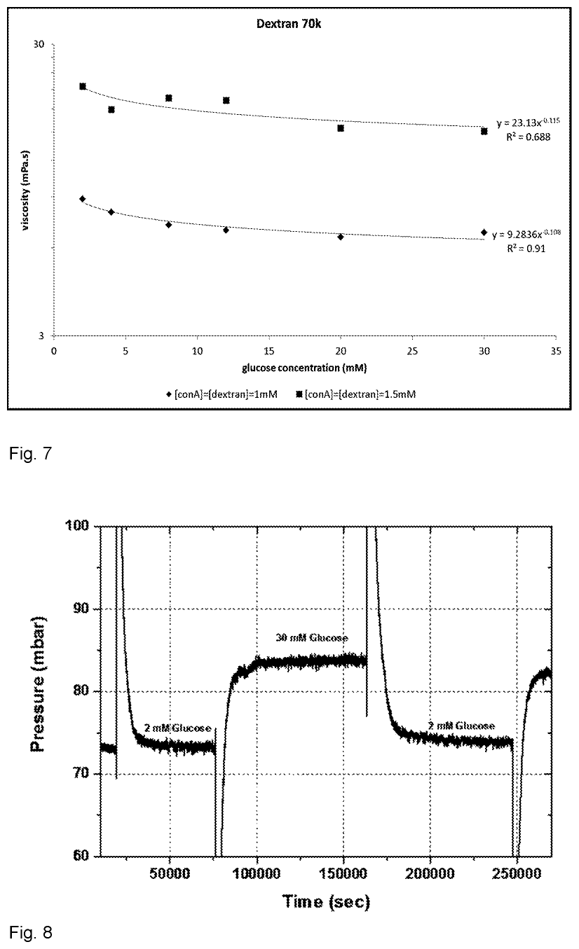

Based on viscosity measurements and simulation results, the inventors found clear trends in term of sensitivities and kinetics of the sensor can be established:
A 1 to 1 molar concentration ratio of ConA to dextran was advantageous with respect to the sensitivities in term of osmotic pressure in the range of glucose concentration [0;30] mM.
A 1 to 1 molar concentration ratio of ConA to dextran was advantageous with respect to fast kinetics and the least asymmetrical response to glucose variations since the viscosity of the active fluid varied very little with the glucose concentration.
At 1 to 1 molar concentration ratio of ConA to dextran, a high ConA concentration (=dextran concentration) gave better sensitivity. However, it was also likely to increase the viscosity of the system, in turn increasing the time response of the sensor.
Experimental studies of the viscosities of different active fluids were performed. These showed a rapid drop (of 3 orders of magnitude) as the concentration of ConA was decreased from 3 mM (based on monomer concentration) to 1 mM. Changes in the concentration of dextran also influenced the viscosity but to a lesser extent. In contrast the molecular weight of the dextran (10 kDa, 40 kDa, 70 kDa) could change the viscosity by orders of magnitude, see FIGS. 6 and 7.
The lower the MW of the dextran, the lower the viscosity was. This means that an active fluid containing dextran 10 kDa will give a faster response than one containing dextran 40 kDa, which in turn is expected to give a faster response than one containing dextran 70 kDa.
Higher MW of the dextran, provided higher differential osmotic pressure of the system. Using a higher size dextran (e.g. 70 kDa) in the active fluid is therefore going to improve the sensitivity of the system.
Simulations of the response time based on these measurements indicated that number of active fluids are expected to have response times (in both increasing a decreasing glucose concentrations) of less than 5 minutes.

The inventors performed a number of experiments to test the response of different active fluids, e.g.:

1.0 mM ConA, 1.0 mM dextran 40 kDa and 1.0 mM ConA, 1.0 mM dextran 70 kDa.
1.5 mM ConA, 1.5 mM dextran 40 kDa and 1.5 mM ConA, 1.5 mM dextran 70 kDa.

### Test of Active Fluids Based on 1.5 mM ConA, 1.5 mM Dextran 40 kDa

Experiments were carried out by cycling the contents of the sample chamber in a macrocell between 2 mM glucose (in a solution containing 100 mM Trizma B buffer, 150 mM NaCl, 10 mM MgCl₂ and 10 mM CaCl₂) and 30 mM glucose in the same solution. At each change of glucose concentration, the solution was removed by hand using a pipette, taking great care not to touch the nanoporous (NP) membrane but bringing the pipette tip as close as possible to the bottom of the sample chamber. The solution was then replaced by the new chosen solution, which was agitated by “pumping” the pipette. The solution was removed and replaced by fresh solution another two times. Temperature was 21° C.

Following this procedure, a reproducible signal was obtained. A change from 2 mM glucose to 30 mM glucose resulted in a large spike down in the measured pressure, followed by a slow rise to a new pressure that was approximately 10 mbar higher than the original value. A change from 30 mM glucose to 2 mM glucose gave a large spike up in measured pressure followed by a slow drift downwards (see FIG. 8). Both the fast spike in one direction and the slow drift in the opposite direction occurred each time.

These two features can be understood when we consider the time course of the events taking place in sample chamber and active fluid chamber, as described below.
1) Starting at low glucose concentration, to begin with, all small solutes are present in equal concentrations on both sides of the NP membrane. Most of the ConA and dextran inside the active fluid chamber is bound together, giving a small overpressure inside the chamber.
2) Glucose is added outside the NP membrane. The concentration of solutes is briefly higher outside the membrane than inside. There is a brief underpressure inside the chamber.
3) The glucose diffuses through the membrane. Small solutes are present in equal concentrations on both sides of the NP membrane. ConA and dextran dissociate and the measured pressure increases to finally give an overpressure inside the chamber.

Thus the observed spike is not an artifact, but shows the process of equilibration of the glucose concentration on the two sides of the membrane.

Results of the study on the active fluids also shows that the present active fluid compositions could be studied for 3 months with no perceptible loss of sensitivity, i.e. long term stability.

Having described preferred embodiments of the invention it will be apparent to those skilled in the art that other embodiments incorporating the concepts may be used. These and other examples of the invention illustrated above are intended by way of example only and the actual scope of the invention is to be determined from the following claims.

1. A composition for use as an active fluid in a continuous glucose sensor, wherein the composition comprises a lectin, a polysaccharide, and at least one chloride salt of a divalent metal ion, wherein the lectin to polysaccharide molar ratio is from 3:1 to 1:1.

2. The composition according to claim 1, wherein the lectin to polysaccharide molar ratio is 3:1, 2:1 or 1:1.

3. The composition according to claim 1, wherein a concentration of the lectin is 0.2-5 mM, 0.5-3 mM or 1-1.5 mM.

4. The composition according to claim 1, wherein a concentration of the polysaccharide is 0.2-5 mM, 0.5-3 mM or 1-1.5 mM.

5. The composition according to claim 1, wherein the polysaccharide has a molecular weight of 10-100 kDa.

6. The composition according to claim 1, wherein the polysaccharide is dextran.

7. The composition according to claim 1, wherein the lectin is Concanavalin A (ConA).

8. The composition according to claim 1, wherein the at least one chloride salt of a divalent metal ion is selected from the group consisting of MgCl₂, CaCl₂) and MnCl₂ or a combination thereof.

9. The composition according to claim 1, wherein the lectin is ConA; the polysaccharide is dextran 40 kDa or dextran 70 kDa; and the lectin to polysacharide molar ratio is 1:1, and the concentration of ConA is 1 or 1.5 mM based on the monomer concentration and the concentration of dextran is 1 or 1.5 mM, wherein the composition further comprises Tris buffer 10 mM, 10 mM MgCl₂, 10 mM CaCl₂, 150 mM NaCl and 30 mM glucose.

10. The composition according to claim 1, wherein the continuous glucose sensor measures the changes in concentrations of glucose fluids in vitro or in vivo by detecting osmotic pressure differences.

11. The composition according to claim 1, wherein the continuous glucose sensor tracks glucose concentrations continuously by recording osmotic pressure, for measuring glucose concentrations in vitro or in vivo.

12. Method for preparing a composition suitable for use as an active fluid in a continuous glucose sensor according to claim 1, comprising:
(i) preparing an aqueous buffer solution having a pH from 7 0 to 7.8, comprising at least one chloride salt of a divalent metal, and optionally glucose;
(ii) either (a) providing a desired amount of the polysaccharide, to which the aqueous buffer solution prepared in (i) is added and left stirring until homogenized and dissolved, and thereafter providing a desired amount of the lectin to which the buffer solution containing said lectin-binding molecule is added and left stirring until homogenized and dissolved, or (b) providing a desired amount of the lectin, to which the aqueous buffer solution prepared in (i) is added and left stirring until homogenized and dissolved, and thereafter providing the desired amount of the polysaccharide, to which the buffer solution containing said lectin is added and left stirring until homogenized and dissolved.
